(19) United States
(12) Patent Application Publication
(10) Pub. No.: US 2022/0218183 A1
(43) Pub. Date: Jul. 14, 2022

(54) SLEEVE SYSTEM FOR SPECULUM AND SPECULUM SYSTEM COMPRISING SAME

(71) Applicant: Ease Medical, Inc., Wilmington, DE (US)

(72) Inventor: Acacia Overstreet, New York, NY (US)

(21) Appl. No.: 17/573,012

(22) Filed: Jan. 11, 2022

Related U.S. Application Data
(60) Provisional application No. 63/135,779, filed on Jan. 11, 2021.

(51) Int. Cl. A61B 1/00; A61B 1/32; A61L 2/04

(52) U.S. Cl. CPC A61B 1/00142; A61L 2202/24; A61L 2/04; A61B 1/32

(57) ABSTRACT

A sleeve system can be used with a speculum having upper and lower blades. A first sleeve can be configured to receive at least a portion of the upper blade of the speculum including an insertional end of the upper blade. A second sleeve can be configured to receive at least a portion of the lower blade of the speculum including an insertional end of the lower blade. The first and second sleeves can be separate elements that are not coupled to each other.

### CROSS-REFERENCE TO RELATED APPLICATION

This application claims priority to and the benefit of the filing date of U.S. Provisional Patent Application No. 63/135,779, filed Jan. 11, 2021, the entirety of which is hereby incorporated by reference herein.

### FIELD

This disclosure relates generally to systems and apparatuses for specula such as vaginal specula.

### BACKGROUND

Conventional pelvic exams involve use of a speculum that is inserted into the vaginal canal to enable a clinician to see therein and perform procedures such as Pap smears. Pelvic exams are notoriously unpleasant by nature. Further, modem specula have changed very little since their conception in the 1840s. To pelvic exam patients, specula can look menacing and medieval. Moreover, because specula are typically made of metal, they feel hard and cold to the touch and are otherwise unpleasant.

Accordingly, there is a need to make the speculum look and feel less unpleasant to the patient, thereby improving the overall experience of the patient.

### SUMMARY

Disclosed herein is a sleeve system configured for use with a speculum. The speculum can have an insertional first end portion, an opposed second end portion, and a longitudinal axis that extends between the first end portion and the second end portion. The speculum can have an upper blade and a lower blade that extend along a longitudinal axis of the speculum. Each of the upper and lower blades can have a respective insertional end and a respective length along a respective axis of elongation. The sleeve system can comprise a first sleeve that is configured to receive at least a portion of the upper blade. Said portion of the upper blade can include the insertional end of the upper blade. A second sleeve can be configured to receive at least a portion of the lower blade. Said portion of the lower blade can include the insertional end of the lower blade. Each of the first and second sleeves can comprise a first end portion that is configured to receive the insertional end of the respective upper or lower blade, an opposed second end portion that defines a respective opening that is configured to receive the respective upper or lower blade therethrough, an outer surface, and an opposed inner surface, wherein the first sleeve is not coupled to the second sleeve.

Also disclosed herein is a speculum system comprising a speculum having an insertional first end portion, an opposed second end portion, and a longitudinal axis that extends between the first end portion and the second end portion. The speculum can have an upper blade and a lower blade that extend along a longitudinal axis of the speculum. Each of the upper and lower blades can have a respective insertional end and a respective length along a respective axis of elongation. A first sleeve can cover at least a portion of the upper blade. Said portion of the upper blade can include the insertional end of the upper blade. A second sleeve can cover at least a portion of the lower blade. Said portion of the lower blade can include the insertional end of the lower blade. Each of the first and second sleeves can comprise a first end portion that covers the insertional end of the respective upper or lower blade, an opposed second end portion that defines a respective opening that is configured to receive the respective upper or lower blade therethrough, an outer surface, and an opposed inner surface.

Also disclosed herein is a method comprising using, during a vaginal procedure, a speculum system comprising a speculum having an insertional first end portion, an opposed second end portion, and a longitudinal axis that extends between the first end portion and the second end portion. The speculum can have an upper blade and a lower blade that extend along a longitudinal axis of the speculum. Each of the upper and lower blades can have a respective insertional end and a respective length along a respective axis of elongation. A first sleeve can cover at least a portion of the upper blade. Said portion of the upper blade can include the insertional end of the upper blade. A second sleeve can cover at least a portion of the lower blade. Said portion of the lower blade can include the insertional end of the lower blade. Each of the first and second sleeves can comprise a first end portion that covers the insertional end of the respective upper or lower blade, an opposed second end portion that defines a respective opening that is configured to receive the respective upper or lower blade therethrough, an outer surface, and an opposed inner surface.

### DESCRIPTION OF THE DRAWINGS

These and other features of the preferred embodiments of the invention will become more apparent in the detailed description in which reference is made to the appended drawings wherein:

FIG. 1 is a perspective view of a speculum system comprising a sleeve system as described herein.

FIG. 2 is an end view of the speculum system from a side opposite the insertional end of the speculum system of FIG. 1.

FIG. 3 is a side view of the speculum system of FIG. 1.

FIG. 4 is a cross sectional view of the speculum system of FIG. 1, taken in the plane 4-4 of FIG. 2.

FIG. 5 is a perspective view of an exemplary sleeve system.

FIG. 6 is a schematic diagram of a cross section of the sleeve system (without the speculum), taken in a plane transverse to the longitudinal axis of the speculum system.

FIG. 7 is a schematic diagram of an end view of an alternative sleeve system that does not have a complementary profile to the inner surfaces of the speculum blades of the speculum as disclosed herein.

FIG. 8 is a side view of an exemplary speculum system as disclosed herein with a first sleeve that covers an entire length of the upper blade of the speculum.

### DETAILED DESCRIPTION

The present disclosure can be understood more readily by reference to the following detailed description of the invention, the figures and the examples included herein.

Before the present apparatuses, systems, and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned or cited herein (including those within the provided reference lists) are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

As used in the specification and the appended claims, the singular forms “a,” “an,” and “the” include the plural referents unless the context clearly dictates otherwise. For example, “a sleeve” should be understood to include one or more sleeves, and so forth.

As used herein, the word “or” can mean any one member of a particular list and, except where otherwise indicated, can also include any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

Optionally, in some aspects, when values are approximated by use of the antecedents “about,” “substantially,” “approximately,” or “generally,” it is contemplated that values within up to 15%, up to 10%, up to 5%, or up to 1% (above or below) of the particularly stated value or characteristic can be included within the scope of those aspects.

As used herein, the term “blade” does not require a profile that is configured for cutting. Rather, the term “blade” should be understood to have its conventional interpretation in the context of a speculum.

As used herein, the term “insertional” refers to the end of a first element that is configured for insertion into a second element. For example, as used herein, an “insertional” end of an element can be configured for insertion into a second element (e.g., a sleeve), with the direction of insertion optionally being toward or generally toward the body of a patient.

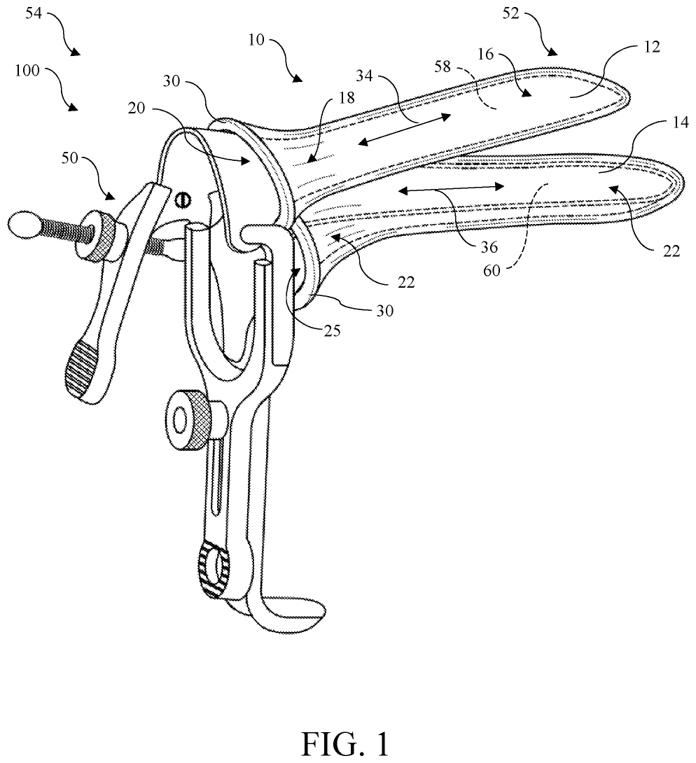

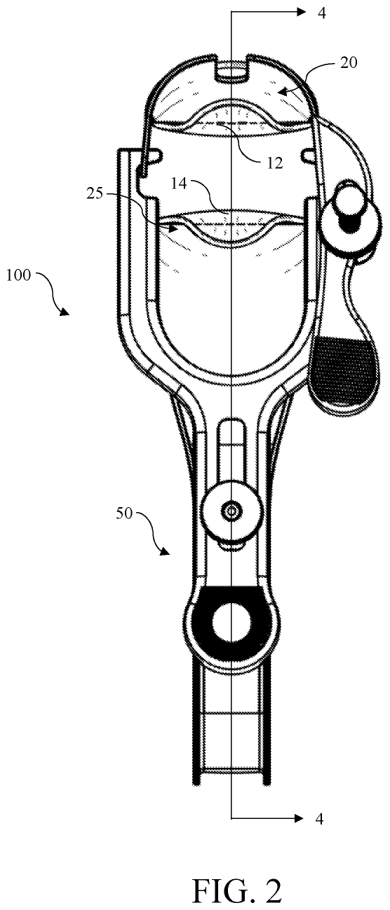

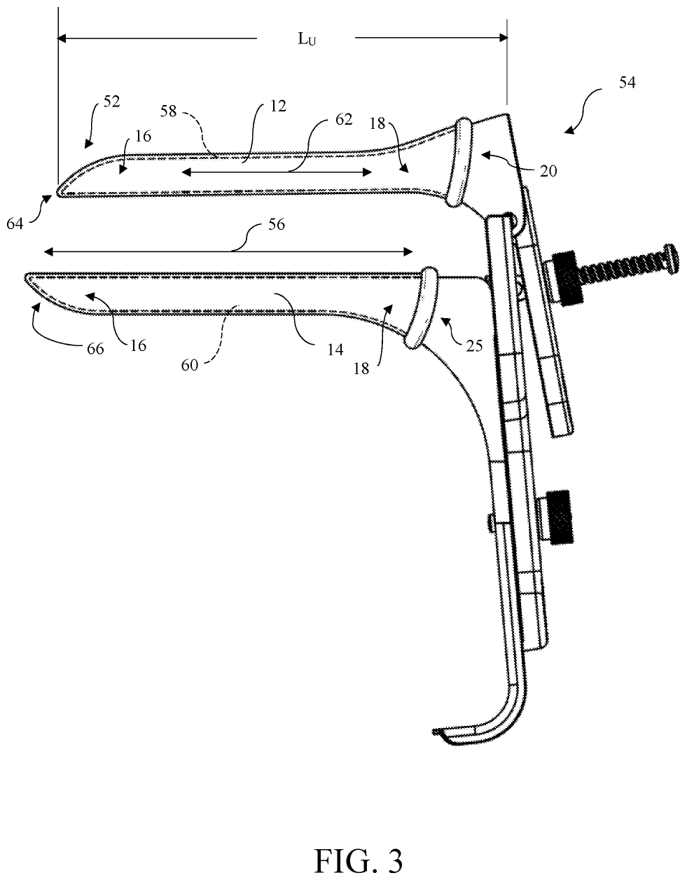

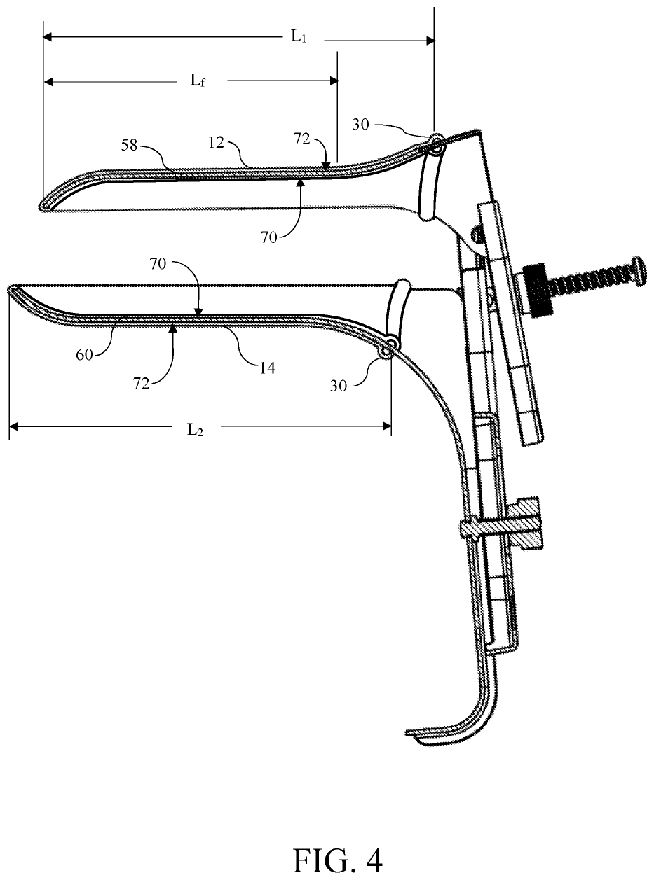

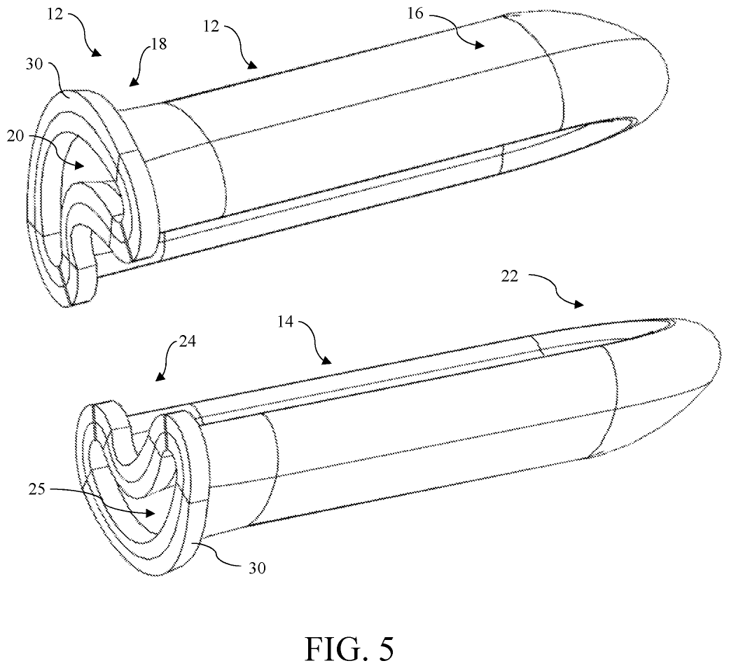

Disclosed herein and with reference to FIGS. 1-5 is a sleeve system 10 for a speculum and a speculum system 100 comprising said sleeve system. The sleeve system 10 can be used with a speculum 50. The speculum 50 can have an insertional first end portion 52, an opposed second end portion 54, and a longitudinal axis 56 that extends between the first end portion and the second end portion. The speculum 50 can have an upper blade 58 and a lower blade 60. The upper blade 58 and lower blade 60 can be articulable relative to each other. For example, the upper and lower blades 58, 60 can be pivotable relative to each other and/or slidable toward and away from each other. The upper blade 58 can extend along an axis of elongation 62 and can have a length Lu along its axis of elongation. The lower blade 60 can be elongate along the longitudinal axis 56 of the speculum. Each of the upper and lower blades 58, 60 can have a respective insertional end 64, 66.

The sleeve system 10 can comprise a first sleeve 12 that is configured to receive at least a portion of the upper blade 58. Said portion of the upper blade 58 can include the insertional end 64 of the upper blade. The sleeve system 10 can comprise a second sleeve 14 that is configured to receive at least a portion of the second blade 60.

The first sleeve 12 can comprise a first portion 16 that is configured to receive therein the insertional end 64 of the upper blade 58 and an opposed second portion 18. The opposed second portion 18 can define an opening 20 that is configured to receive therethrough the insertional end 64 of the upper blade 58.

The second sleeve 14 can comprise a first portion 22 that is configured to receive therein the insertional end 66 of the lower blade 60 and an opposed second portion 24. The opposed second portion 24 can define an opening 25 that is configured to receive therethrough the insertional end 66 of the lower blade 60.

Each of the first and second sleeves 12, 14 can have a respective outer surface 26 and an opposed inner surface 28.

In some aspects, the first and second sleeves 12, 14 can be separate elements that are not coupled to each other.

In exemplary aspects, the sleeves can be retained on their respective blades via friction. In some optional aspects, the second portion 18, 24 of the respective first and second sleeves 12, 14 can have an inner surface that is tighter against the blade than the respective first portions 16, 22. In further aspects, the sleeves can be tight against their respective blades along all, or substantially all, of their respective lengths. Accordingly, in some aspects, during a vaginal procedure, the sleeve system 10 can stay in position relative to the speculum. For example, the sleeve system 10 can be configured not to slide, bunch, or wrinkle during movement relative to the vagina. It is contemplated that the material(s) of the sleeve system (e.g., silicone), as well as the sizing of the sleeves relative to the speculum, can inhibit movement (e.g. sliding, bunching, or wrinkling) relative to the speculum.

In some optional aspects, the first and second sleeves 12, 14 can comprise polymer. For example, the first and second sleeves 12, 14 can comprise silicone. In some aspects, the silicone can be nonporous. It is contemplated that polymer such as silicone can prove a softer feel as compared to a metal speculum. Still further, such a polymer can feel warmer than a metal speculum. It is contemplated that silicone is a material that is familiar to many women (due to the presence of silicone in intimacy products and sexual stimulation products). Accordingly, the sleeve system can improve the emotional comfort level experienced by the patient (e.g., avoiding the jarring experience of both seeing and feeling metal).

In various aspects, the polymer can have a modulus at 100% elongation (modulus 100%) from about 32 pounds per square inch (PSI) to about 580 PSI (e.g., from about 70 PSI to about 340 PSI). In optional aspects, the polymer can have a tear strength from about 45 pounds per inch (PPI) to about 290 PPI (e.g., from about 150 to about 290 PPI). In optional aspects, the polymer can have a shore A hardness from about 10 to about 70 (e.g., from about 20 to about 50). In some optional aspects, the first and second sleeves 12, 14 can be configured for heat sterilization. Accordingly, optionally, the first and seconds sleeves 12, 14 can be configured for heating to at least 121° C. or at least 132° C. Accordingly, the sleeve system 10 can be reusable. In exemplary aspects, the polymer can be configured to elastically deform. For example, the polymer can be configured to elongate. In this way, the first and second sleeves can expand to receive the respective blades therein.

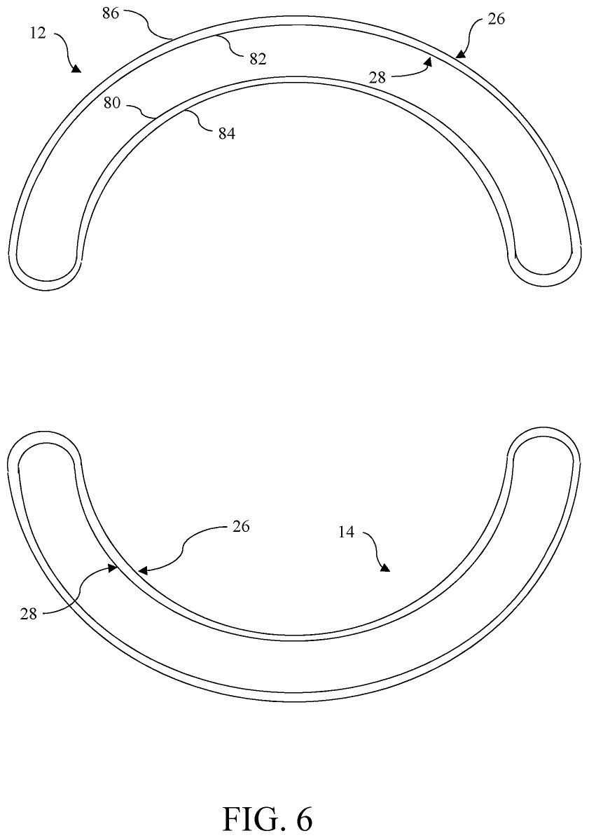

Referring to FIG. 6, in some aspects, each of the first and second sleeves 12, 14 can define a respective wall thickness, t, between the outer surface 26 and the inner surface 28. In some aspects, said thickness can be from about 0.1 mm to about 1.5 mm, or from about 0.2 mm to about 1.5 mm, or from about 0.5 mm to about 1.5 mm, or at least 0.1 mm, or at least 0.2 mm, or at least 0.3 mm at least 0.4 mm, or at least 0.5 mm, or at least 0.6 mm, or at least 0.7 mm, or at least 0.8 mm, or at least 0.9 mm, or at least 1 mm or at least 1.1 mm. Optionally, the wall thickness, t, of each sleeve can be substantially consistent along an entirety, or substantially an entirety, of the respective length of the sleeve.

Still further, in some aspects, the first and second sleeves can have a thermal conductivity from about 0.1 W/m-K to about 0.4 W/m-K. Accordingly, the thermal conductivity of the material and the thickness of the first and second sleeves 12, 14 can cooperate to define a thermal resistance that insulates the patient from the speculum so that the speculum system does not feel unpleasantly cold when inserted into the patient.

In some optional aspects, the one or both of the first and second sleeves 12, 14 can have a lip 30 that extends peripherally around the respective opening 20, 24 and extends radially outwardly. Optionally, the lip 30 can be defined by an increased wall thickness of the respective sleeve at the opening. In further aspects, the lip 30 can be defined by a shaped portion of the sleeve that is formed to radially extend from the outer surface of the respective blade. That is, both the outer and inner surfaces 26, 28 of the sleeve at the lip 30 can extend away from the respective upper or lower blade. Optionally, said shaped portion can have a rounded (e.g., semicircular) cross-sectional shape around the sleeve. In further aspects, the lip 30 can be defined by a widened material thickness. In still further aspects, the lip 30 can have any suitable shape, including a radially extending flare. In exemplary aspects, the lip 30 can be used by a clinician to grip the respective sleeve during insertion of the blade into the sleeve. In further aspects, the lip 30 can serve as a grip for the clinician that can facilitate removal of the sleeve from the speculum. In some optional aspects, the lip 30 can be configured to be tight against the respective blade in order to retain the respective sleeve on the blade. For example, prior to insertion of the respective blade into the sleeve, the sleeve can have a reduced inner perimeter at the lip. For example, around some or all of the lip 30, the sleeve can extend inwardly relative to portions of the sleeve toward the first end of the sleeve. In this way, with the blade inserted therein, the lip 30 can elastically expand to receive the blade.

In some aspects, the first and second sleeves 12, 14 can be opaque. In this way, the metallic look of the speculum can be obscured. Optionally, the first and second sleeves 12, 14 can comprise a colorant. In this way, the first and second sleeves can be colored to have a more appealing look. For example, the first and second sleeves, can be pink, red, white, gray, blue, green, gold, or any desired color. Optionally, the first and second sleeves can have a glossy outer surface. In further aspects, the first and second sleeves can have a matte outer surface. Optionally the first and second sleeves can be multicolored. Optionally, the first and second sleeves can be differently colored. Optionally, the color of the sleeves can indicate the particular speculum or specula (e.g., size or shape) with which they are compatible. Thus, for example, different sizes (e.g., small, medium, and large) can be different colors.

In some aspects, the first and second sleeves 12, 14 can have sufficient wall thickness and rigidity that, without the blades of the speculum are inserted therein, the inner surfaces of the sleeves generally have the contour of the blades. Thus, the first and second sleeves can be complementarily shaped to the respective portions of the upper and lower blades received therein. For example, referring to FIG. 6, the inner surface 28 (and the outer surface 26) of the first sleeve 12, in cross sections along its length in planes perpendicular to the axis of elongation 62, can be generally U-shaped. Further, in some aspects, the inner surface 28 (and the outer surface 26) of the second sleeve 14, in cross sections along its length in planes perpendicular to its axis of elongation (e.g., optionally, the longitudinal axis 56), can be generally U-shaped. For example, inner surfaces 28 of the first and second sleeves 12, 14 can each have a convex portion 80 and an opposed concave portion 82. The outer surfaces 26 of the first and second sleeves 12, 14 can likewise have corresponding opposed concave and convex portions 84, 86. Accordingly, in some optional aspects, with the upper and lower blades 58, 60 inserted respectively in to the first and second sleeves, 12, 14, the first and second sleeves can be configured to contact both inner surfaces 70 and outer surfaces 72 of the respective upper and lower blades. In this way, the first and second sleeves, 12, 14 have a profile that corresponds to or generally matches the surface profiles of the blades such that the sleeves 12, 14 do not block the view of the clinician. In some optional aspects, the first and second sleeves 12, 14 can be sufficiently flexible that the sleeves have a generally undefined cross-sectional profile until the blades of the speculum are inserted therein. Alternatively, in other aspects, the first and second sleeves 12, 14 can have a predefined cross-sectional profile (prior to insertion of the blades) that matches or generally matches the contour of the blades.

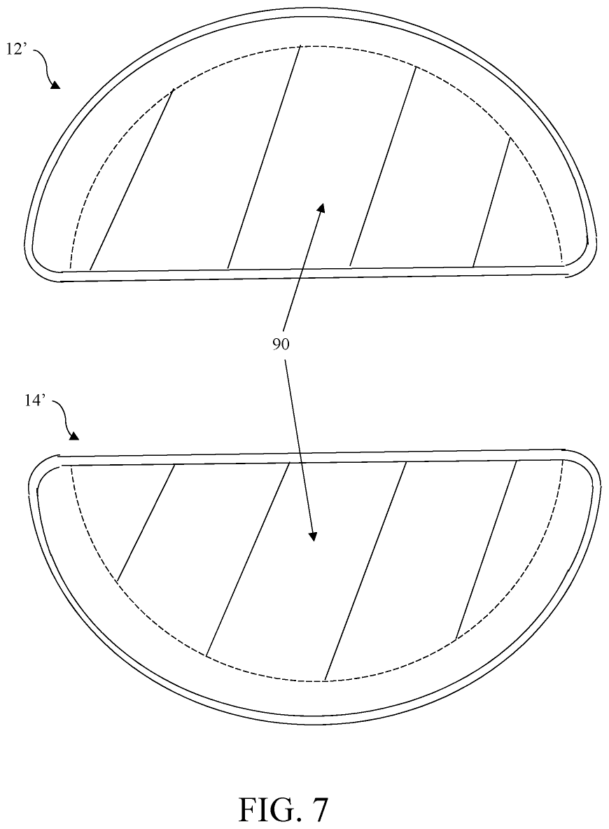

In alternative aspects, and as illustrated schematically in FIG. 7, it is contemplated that the sleeve system can comprise sleeves 12′, 14′ that do not contact or generally trace the inner surface 70 of the blades such that the sleeves block portions 90 of the view of clinician. In further optional aspects, the first and second sleeves 12′, 14′ can be sufficiently flexible that the sleeves have a generally undefined cross-sectional profile until the blades of the speculum are inserted therein. For example, in some optional aspects, and as illustrated in FIG. 7, the sleeves 12′, 14′ can merely contour to portions (e.g., outer portions) of the surfaces of the blades and do not have a defined cross sectional profile. Accordingly, the sleeves 12′, 14′ can be configured to extend transversely between opposed longitudinally extending edges of the respective upper and lower blades (rather than following the profile of the inner surfaces 70 of the upper and lower blades). For example, in some aspects, the sleeves 12′, 14′ can stretch between the longitudinally extending edges of the respective upper and lower blades (e.g., so that portions of the sleeves extend generally parallel to a transverse axis that is perpendicular to the longitudinal axis 56).

The first sleeve 12 can have an axis of elongation 34 and a length L₁ along the axis of elongation 34. The second sleeve 14 can have an axis of elongation 36 and a length L₂ along the axis of elongation 36. In some aspects, the speculum 50 can have a generally flat portion 76 and can flare outwardly at a length L_{f} from the insertional end. In some aspects, each of the first and second sleeves 12, 14 can have respective lengths L₁, L₂ that are greater than the length L_{f}. For example, the respective lengths L₁, L₂ of the first and second sleeves 12, 14 can each be greater than 50% or greater than 60% or greater than 65% or greater than 75% or greater than 80% or greater than 85% or greater than 90% or greater than 95% or substantially the same length as the length of the upper blade 58. In this way, the first and second sleeves 12, 14 can be configured to cover an entirety of the speculum 50 that is inserted into the patient. optionally, the respective lengths L₁, L₂ of the first and second sleeves 12, 14 can each be less than 95%, or less than 90%, or less than 85% of the length of the upper blade so that the sleeves are spaced from, and do not interfere with, a hinged coupling between the upper and lower blades. Optionally, the first and second sleeves 12, 14 can be configured to stretch and elongate upon receipt of the respective upper and lower blades 58, 60. In these aspects, the first and second sleeves can have a respective resting length (prior to stretching or other deformation to receive the upper and lower blades) that is less than the respective lengths of the first and second sleeves L₁, L₂ with the blades received therein. In some aspects, said resting lengths of the first and second blades can be within 5%, or within 3%, or within 2%, or within 1% of the respective lengths L₁, L₂ of the sleeves with the blades received therein.

The speculum 50 can be, for example, a Graves speculum or a Pederson speculum. Accordingly, the sleeve system 10 described herein can be sized and shaped to cooperate with dimensions of a particular speculum. In yet further aspects, the sleeve system can be flexible and resilient so that the first and second sleeves 12, 14 can expand to fit specula blades of different dimensions. In exemplary, optional aspects, the first and second sleeves can have a width from about 0.5 inches to about 1.5 inches (e.g., about 0.5 inches). In some optional aspects, each of the first and second sleeves 12, 14 can have length of about 3 inches to about 6 inches (e.g., about 3 inches, about 4 inches, about 5 inches). Optionally, the first and second sleeves 12, 14 can have the same or approximately the same length and width. In further aspects, the length and/or width of the first and second sleeves can differ. Optionally, the first and second sleeves 12, 14 can have the same shape prior to receipt of the respective blades therein.

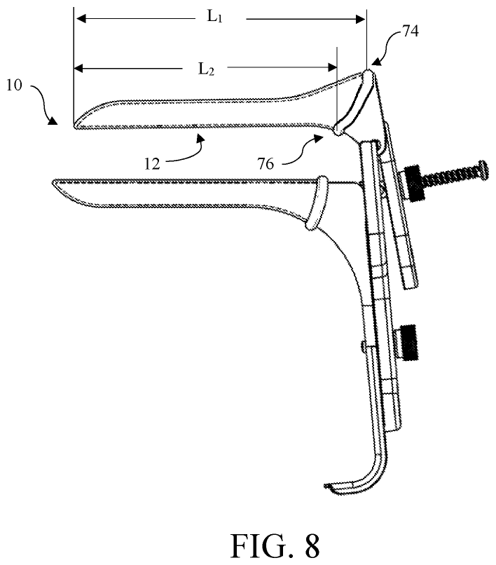

Referring also to FIG. 8, the upper sleeve can have a top end 74 and a bottom end 76 at the opening 20. Optionally, the top end of the upper sleeve 12 can have a first length L₁ (measured from the first end portion of the upper sleeve), and the bottom end of the upper sleeve can have a second length L₂ (measured from the first end portion of the upper sleeve) that is less than the first length. Optionally, in these aspects, the top end 74 of the sleeve can extend to the end of the upper blade 12 opposite the insertional end.

Optionally, each of the first and second sleeves 12, 14 can stretch to receive the cross sectional profile respective upper and lower blades 58, 60. For example, prior to receipt of the respective upper or lower blade therein, the respective inner surfaces of each of the first and second sleeves, in cross section in planes perpendicular to the respective longitudinal sleeve axis of elongation 34, 36, can have cross sectional areas that are less than the cross sectional area of the corresponding portion of the respective speculum blade received therein. Accordingly, some or all of the sleeve can elastically expand to receive the blade therein.

In further aspects, the first and second sleeves 12, 14 do not stretch to receive the cross sectional profile of the upper and lower blades 58, 60. For example, prior to receipt of the respective upper or lower blade therein, the respective inner surfaces of each of the first and second sleeves, in cross section in planes perpendicular to the respective longitudinal sleeve axis of elongation 34, 36, can have cross sectional areas that are greater than the cross sectional area of the corresponding portion of the respective speculum blade received therein.

In still further aspects, each of the first and second sleeves can have equal or substantially equal cross sectional areas to the cross sectional areas of the corresponding portions of the respective speculum blade received therein.

The speculum 50 can be a vaginal speculum. In further aspects, speculum can be an anal speculum or a nasal speculum. Accordingly, the sleeve system 10 can be configured for use with (e.g., sized for use with) the anal or nasal speculum. In some aspects, the speculum 50 can comprise metal. For example, the speculum 50 can comprise almost entirely metal. In further aspects, the speculum 50 can comprise polymer. For example, the speculum 50 can comprise almost entirely polymer. Although embodiments herein are described as having upper and lower blades, it should be understood that further embodiments can be used with side-by-side blades, such as those of an anal speculum. In yet further aspects, the sleeve system 10 can comprise three or more sleeves for respective receipt of each blade of a speculum comprising three or more sleeves.

A method can comprise positioning the first and second sleeves 12, 14 on the upper and lower blades 58, 60 of the speculum 50. For example, the upper blade 58 can be inserted into the first sleeve 12 and the lower blade 60 can be inserted into the second sleeve 14. The upper and lower blades 58, 60 can optionally be inserted until their respective insertional ends 64, 66 reach and contact the end of the respective sleeve opposing the openings 20, 25.

A method can comprise using the speculum system 100 during a vaginal procedure (e.g., a pelvic examination). The method can further comprise removing the first and second sleeves 10, 12 from the respective upper and lower blades 58, 60 of the speculum 50. In some aspects, the first and second sleeves 10, 12 can be sterilized once removed from the speculum. The method can further comprise positioning the first and second sleeves 12, 14 on the upper and lower blades 58, 60 of the same or a different speculum 50. In further aspects, the first and second sleeves 10, 12 can be sterilized while on the speculum (with the respective blades received therein). Accordingly, the sleeve system 10 can be reused for another vaginal procedure (e.g., after sterilization).

In some exemplary aspects, a kit can comprise a plurality of sets of sleeves. For example, a kit can comprise a plurality of sets of sleeves that are configured to fit different sizes and/or shapes of specula 50. In further aspects, a kit can comprise a speculum 50 and a speculum system 10 that is configured for use with said speculum. Optionally, a kit can further comprise a sleeve system 10 and a lubricant (e.g., a vaginal lubricant).

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A sleeve system configured for use with a speculum, the speculum having an insertional first end portion, an opposed second end portion, and a longitudinal axis that extends between the first end portion and the second end portion, the speculum having an upper blade and a lower blade that extend along a longitudinal axis of the speculum, each of the upper and lower blades having a respective insertional end and a respective length along a respective axis of elongation, the sleeve system comprising:

a first sleeve that is configured to receive at least a portion of the upper blade, wherein the portion of the upper blade includes the insertional end of the upper blade; and

a second sleeve that is configured to receive at least a portion of the lower blade, wherein the portion of the lower blade includes the insertional end of the lower blade,
wherein each of the first and second sleeves comprises:
   a first end portion that is configured to receive the insertional end of the respective upper or lower blade;
   an opposed second end portion that defines a respective opening that is configured to receive the respective upper or lower blade therethrough;
   an outer surface; and
   an opposed inner surface,

Aspect 2: The sleeve system of aspect 1, wherein the first sleeve is not coupled to the second sleeve.

Aspect 3: The sleeve system aspect 1 or aspect 2, wherein the first and second sleeve comprise polymer.

Aspect 4: The sleeve system of aspect 3, wherein the first and second sleeve comprise silicone.

Aspect 5: The sleeve system of aspect 3 or aspect 4, wherein the polymer has a modulus of 100% elongation from about 32 pounds per square inch (PSI) to about 580 PSI.

Aspect 6: The sleeve system of any one of aspects 3-5, wherein the polymer has a tear strength from about 45 to about 290 pounds per inch.

Aspect 7: The sleeve system of any one of aspects 3-6, wherein the polymer has a shore A hardness from about 10 to about 70.

Aspect 8: The sleeve system of any one of the preceding aspects, wherein each of the first and second sleeves defines a respective wall thickness between the outer surface and the inner surface, wherein the wall thickness is at least 0.2 mm along an entire length of said sleeve.

Aspect 9: The sleeve system of aspect 8, wherein the wall thickness of each sleeve is substantially consistent along substantially an entirety of the respective length of said sleeve.

Aspect 10: The sleeve system of any one of the preceding aspects, wherein each of the first and second sleeves has a lip that extends peripherally around the opening.

Aspect 11: The sleeve system of any one of the preceding aspects, wherein the first and second sleeves comprise a colorant.

Aspect 12: The sleeve system of any one of the preceding aspects, wherein the first and second sleeves are opaque.

Aspect 13: The sleeve system of any one of the preceding aspects, wherein each of the first and second sleeves extends along a respective longitudinal sleeve axis, wherein the respective inner surface of each of the first and second sleeves, in cross section in planes perpendicular to the respective longitudinal sleeve axis, is generally U-shaped.

Aspect 14: The sleeve system of any one of the preceding aspects, wherein the upper and lower blades have respective inner and outer surfaces, wherein the first and second sleeves are configured to contact both the inner and outer surfaces of the respective upper or lower blade.

Aspect 15: The sleeve system of any one of the preceding aspects, wherein the first and second sleeves are configured for heat sterilization.

Aspect 16: A speculum system comprising:

a speculum having an insertional first end portion, an opposed second end portion, and a longitudinal axis that extends between the first end portion and the second end portion, the speculum having an upper blade and a lower blade that extend along a longitudinal axis of the speculum, each of the upper and lower blades having a respective insertional end and a respective length along a respective axis of elongation;

a first sleeve covering at least a portion of the upper blade, wherein the portion of the upper blade includes the insertional end of the upper blade; and

a second sleeve covering at least a portion of the lower blade, wherein the portion of the lower blade includes the insertional end of the lower blade,

wherein each of the first and second sleeves comprises:
a first end portion that covers the insertional end of the respective upper or lower blade;
an opposed second end portion that defines a respective opening that is configured to receive the respective upper or lower blade therethrough;
an outer surface; and
an opposed inner surface.

Aspect 17: The speculum system of aspect 16, wherein the speculum has an outward flare at a first length from the insertional end of the speculum, wherein the first and second sleeves each have a length along a respective axis of elongation that is greater than the first length.

Aspect 18: The sleeve system of aspect 16 or aspect 17, wherein each of the first and second sleeve each have a length along a respective axis of elongation that is greater than 65 percent of the length of the upper blade.

Aspect 19: The sleeve system of any one of aspects 16-18, wherein the first sleeve is configured to cover at least 95% of the length of the upper blade.

Aspect 20: The sleeve system of any one of aspects 16-19, wherein the respective lengths of the first and second sleeves are each less than 90% of the length of the upper blade.

Aspect 21: The sleeve system of any one of aspects 16-20, wherein each of the first and second sleeves extends along a respective longitudinal sleeve axis, wherein, prior to receipt of the respective upper or lower blade therein, the respective inner surfaces of each of the first and second sleeves, in cross section in planes perpendicular to the respective longitudinal sleeve axis, have cross sectional areas that are less than the cross sectional area of the corresponding portion of the respective speculum blade received therein.

Aspect 22: The sleeve system of any one of aspects 16-20, wherein each of the first and second sleeves extends along a respective longitudinal sleeve axis, wherein, prior to receipt of the respective upper or lower blade therein, the respective inner surface of each of the first and second sleeves, in cross section in planes perpendicular to the respective longitudinal sleeve axis, have cross sectional areas that are greater than the cross sectional area of the corresponding portion of the respective speculum blade received therein.

Aspect 23: The sleeve system of any one of aspects 16-22, wherein the inner surfaces of the first and second sleeves are complementarily shaped to the respective portions of the upper and lower blades received therein.

Aspect 24: A method comprising:
using, during a vaginal procedure, a speculum system comprising:
   a speculum having an insertional first end portion, an opposed second end portion, and a longitudinal axis that extends between the first end portion and the second end portion, the speculum having an upper blade and a lower blade that extend along a longitudinal axis of the speculum, each of the upper and lower blades having a respective insertional end and a respective length along a respective axis of elongation;
   a first sleeve covering at least a portion of the upper blade, wherein the portion of the upper blade includes the insertional end of the upper blade; and
   a second sleeve covering at least a portion of the lower blade, wherein the portion of the lower blade includes the insertional end of the lower blade,
wherein each of the first and second sleeves comprises:
   a first end portion that covers the insertional end of the respective upper or lower blade;
   an opposed second end portion that defines a respective opening that is configured to receive the respective upper or lower blade therethrough;
   an outer surface; and
   an opposed inner surface.

Aspect 25: The method of aspect 24, further comprising:
removing the first and second sleeves from the respective upper and lower blades of the speculum; and
sterilizing the first and second sleeves.

Aspect 26: The method of aspect 25, further comprising: positioning the first and second sleeves on upper and lower blades of the same or a different speculum.

Aspect 27: The method of aspect 24, further comprising sterilizing the speculum system with the first sleeve covering the at least a portion of the upper blade and the second sleeve covering the at least a portion of the lower blade.

Aspect 28: The method of aspect 24, further comprising: after performing the vaginal procedure, sterilizing the first and second sleeves.

Although the foregoing invention has been described in some detail by way of illustration and example for purposes of clarity of understanding, certain changes and modifications may be practiced within the scope of the appended claims.

What is claimed is:

1. A sleeve system configured for use with a speculum, the speculum having an insertional first end portion, an opposed second end portion, and a longitudinal axis that extends between the first end portion and the second end portion, the speculum having an upper blade and a lower blade that extend along a longitudinal axis of the speculum, each of the upper and lower blades having a respective insertional end and a respective length along a respective axis of elongation, the sleeve system comprising:
a first sleeve that is configured to receive at least a portion of the upper blade, wherein the portion of the upper blade includes the insertional end of the upper blade; and
a second sleeve that is configured to receive at least a portion of the lower blade, wherein the portion of the lower blade includes the insertional end of the lower blade,
wherein each of the first and second sleeves comprises:
a first end portion that is configured to receive the insertional end of the respective upper or lower blade;
an opposed second end portion that defines a respective opening that is configured to receive the respective upper or lower blade therethrough;
an outer surface; and
an opposed inner surface, wherein the first sleeve is not coupled to the second sleeve.

2. The sleeve system claim 1, wherein the first and second sleeve comprise polymer.

3. The sleeve system of claim 2, wherein the first and second sleeve comprise silicone.

4. The sleeve system of claim 2, wherein the polymer has a modulus of 100% elongation from about 32 pounds per square inch (PSI) to about 580 PSI.

5. The sleeve system of claim 2, wherein the polymer has a shore A hardness from about 10 to about 70.

6. The sleeve system of claim 1, wherein each of the first and second sleeves defines a respective wall thickness between the outer surface and the inner surface, wherein the wall thickness is at least 0.2 mm along an entire length of said sleeve.

7. The sleeve system of claim 6, wherein the wall thickness of each sleeve is substantially consistent along substantially an entirety of the respective length of said sleeve.

8. The sleeve system of claim 1, wherein each of the first and second sleeves has a lip that extends peripherally around the opening.

9. The sleeve system of claim 1, wherein the first and second sleeves comprise a colorant.

10. The sleeve system of claim 1, wherein the first and second sleeves are opaque.

11. The sleeve system of claim 1, wherein each of the first and second sleeves extends along a respective longitudinal sleeve axis, wherein the respective inner surface of each of the first and second sleeves, in cross section in planes perpendicular to the respective longitudinal sleeve axis, is generally U-shaped.

12. The sleeve system of claim 1, wherein the upper and lower blades have respective inner and outer surfaces, wherein the first and second sleeves are configured to contact both the inner and outer surfaces of the respective upper or lower blade.

13. The sleeve system of claim 1, wherein the first and second sleeves are configured for heat sterilization.

14. A speculum system comprising:
a speculum having an insertional first end portion, an opposed second end portion, and a longitudinal axis that extends between the first end portion and the second end portion, the speculum having an upper blade and a lower blade that extend along a longitudinal axis of the speculum, each of the upper and lower blades having a respective insertional end and a respective length along a respective axis of elongation;
a first sleeve covering at least a portion of the upper blade, wherein the portion of the upper blade includes the insertional end of the upper blade; and
a second sleeve covering at least a portion of the lower blade, wherein the portion of the lower blade includes the insertional end of the lower blade,
wherein each of the first and second sleeves comprises:
a first end portion that covers the insertional end of the respective upper or lower blade;
an opposed second end portion that defines a respective opening that is configured to receive the respective upper or lower blade therethrough;
an outer surface; and
an opposed inner surface.

15. The sleeve system of claim 14, wherein each of the first and second sleeve each have a respective length along a respective axis of elongation that is greater than 65 percent of the length of the upper blade.

16. The sleeve system of claim 14, wherein the first sleeve covers at least 95% of the length of the upper blade.

17. The sleeve system of claim 14, wherein the respective lengths of the first and second sleeves are each less than 90% of the length of the upper blade.

18. The sleeve system of claim 14, wherein the inner surfaces of the first and second sleeves are complementarily shaped to the respective portions of the upper and lower blades received therein.

19. A method comprising:
using, during a vaginal procedure, a speculum system comprising:
a speculum having an insertional first end portion, an opposed second end portion, and a longitudinal axis that extends between the first end portion and the second end portion, the speculum having an upper blade and a lower blade that extend along a longitudinal axis of the speculum, each of the upper and lower blades having a respective insertional end and a respective length along the longitudinal axis of the speculum;
a first sleeve covering at least a portion of the upper blade, wherein the portion of the upper blade includes the insertional end of the upper blade; and
a second sleeve covering at least a portion of the lower blade, wherein the portion of the lower blade includes the insertional end of the lower blade,
wherein each of the first and second sleeves comprises:
a first end portion that covers the insertional end of the respective upper or lower blade is received;
an opposed second end portion that defines a respective opening that is configured to receive the respective upper or lower blade therethrough;
an outer surface; and
an opposed inner surface.

20. The method of claim 19, further comprising:
after performing the vaginal procedure, sterilizing the first and second sleeves.
